(19) United States
(12) Patent Application Publication
(10) Pub. No.: US 2021/0210192 A1
(43) Pub. Date: Jul. 8, 2021

(54) SYSTEM AND METHOD FOR ASSESSING AND TREATING DYSMENORRHEA

(71) Applicant: ZenChi, Inc., New York, NY (US)

(72) Inventor: Lulu Ge, New York, NY (US)

(73) Assignee: ZenChi, Inc., New York, NY (US)

(21) Appl. No.: 17/142,504

(22) Filed: Jan. 6, 2021

Related U.S. Application Data
(60) Provisional application No. 62/958,080, filed on Jan. 7, 2020.

(51) Int. Cl. G16H 20/90; A61B 5/00; A61K 36/505; A61K 36/236; A61K 36/65; A61K 36/076; A61K 36/232; A61K 36/286; A61K 36/484; A61K 36/539; A61K 36/284; A61K 36/534; A61K 36/906; A61K 36/744; A61K 36/233; A61K 36/8905; A61K 36/533; A61K 36/58; A61K 36/53; G16H 10/20; G16H 50/20

(52) U.S. Cl. CPC G16H 20/90; G16H 50/20; A61B 5/4854; A61B 5/4318; A61K 36/505; A61K 36/236; A61K 36/65; A61K 36/076; A61K 36/232; A61K 36/286; A61K 36/484; A61K 36/539; A61K 36/284; A61K 36/534; A61K 36/906; A61K 36/744; A61K 36/233; A61K 36/8905; A61K 36/533; A61K 36/58; A61K 36/53; G16H 10/20; A61B 5/0088

(57) ABSTRACT

A computer implemented method and system are provided. Such a method includes collecting health data from a female subject, assessing hormonal irregularities based on the health data to identify a pattern of hormonal imbalance, and prescribing a personalized herbal formula based on the pattern of hormonal imbalance to prevent or treat a condition or disorder related to a menstrual cycle of the female subject. Health data, which may be collected through online assessment, may include self-reported answers to a list of questions, and one or more photos of tongue of the female subject.

### PRIORITY CLAIM AND CROSS-REFERENCE

This application claims the benefit of U.S. Provisional Application No. 62/958,080, filed Jan. 7, 2020, which application is expressly incorporated by reference herein in its entirety.

### FIELD OF THE INVENTION

The disclosure relates to a system and a method for preventing and treating conditions or disorders related to women's menstrual cycles generally. More particularly, the disclosed subject matter relates to a software program, a system, and a method for assessing, preventing, and/or treating dysmenorrhea.

### BACKGROUND

Currently up to 90% of all women in the U.S. experience menstrual pain and/or discomfort. This problem affects such a large percentage of the population and leads to significant economic loss. It is estimated that up to 32% of women miss work or school due to menstrual cycle discomfort. The related pain also causes a significant decrease in quality of life of the women who suffer. However, few effective solutions are available for preventing and healing the disorder or discomfort.

Drugs are used as current solutions for the problem of dysmenorrhea. Currently the majority of consumers use nonsteroidal anti-inflammatory drugs (NSAIDS) or hormonal contraceptives as the primary ways to remedy menstrual symptoms. It's been documented that 58% of people on hormonal contraceptives (e.g., birth control pills) use them for menstrual symptom relief. But these solutions have been linked to long-term negative health consequences.

Synthetic hormones in the combined oral contraceptive pill have been used in an attempt to manage dysmenorrhea. These hormones act by suppressing ovulation and lessening the development of endometrial lining of the uterus. In this manner, menstrual fluid volume decreases along with the amount of rostaglandins produced, which then reduces dysmenorrhea by decreasing uterine motility, and uterine cramping.

A 2009 Cochrane review of ten randomized controlled trials (RCTs) found that there is limited evidence for pain improvement as there was little to no relief in symptoms with the use of the combined oral contraceptive pill in women with dysmenorrhea and that there is no evidence of a difference between different oral contraceptive pill (OCP) preparations (different levels of estrogen/progesterone). Even when there is supportive evidence, studies of this type of therapy point out that any relief or reduction in prostaglandins are confined only to that particular cycle, with no carry-over effect after stopping the OCP.

That being said, this type of long-term hormonal/endocrine therapy is only potentially useful if long-term contraception is also desired. In 2011, the Guttmacher Institute estimated that 1.5 million women in the U.S. are using oral contraception for non-contraceptive purposes. More than half of pill users, 58%, rely on the method, at least in part for purposes other than pregnancy prevention. Thirty-one percent use it for cramps or menstrual pain, 28% for menstrual regulation.

Because of the high prevalence of dysmenorrhea in adolescent girls, a high population are immediately put on oral contraceptives. 82% of 15-19-year-olds who use oral contraceptive pills (OCPs) say they do so for non-contraceptive reasons. Many stay on OCPs indefinitely, and many times until they are ready to conceive, which can be in excess of 10-20 years, and use is only expected to grow. However, there are many known side effects of OCPs:

decreased fertility, depression, increased risk of breast cancer, reduced sexual desire, increased risk of venous thromboembolism, weight gain, nausea, and even adverse environmental effects.

Other pharmacological therapies include drugs that inhibit prostaglandins, such as non-steroidal anti-inflammatory drugs (NSAIDs). There are many types of NSAIDs widely used as analgesics and anti-inflammatory agent. This can be achieved through inhibiting cyclooxygenase (COX) enzymes including COX-1 and COX-242 or by reducing myometrial activity (contraction of the uterus) through inhibition of the prostaglandin F2 (PGF2) synthesis and reducing vasopressin secretion, which may effectively reduce menstrual pain. These drugs provide no long-term relief as the treatment relieves symptoms on an episode-by-episode basis only. The failure rate of NSAIDs can be upwards of 75% and these drugs may be contraindicated or not tolerated by some women. Side effects are mainly gastrointestinal, and cardiovascular.

Other treatment modalities for dysmenorrhea includes surgical interruption of the pelvic nerve pathways, heat therapy, moderate exercise, specific yoga poses, acupressure, or an acupressure device known as the Relief Brief, or acupuncture.

Overall, primary dysmenorrhea causes great loss in personal health and wellbeing, along with having to take time off work or school which has also resulted in loss of productivity, eventually leading to economic loss. Because current accepted management practices (namely contraceptive and pharmaceutical pain management) are not well tolerated and also not permanent solutions, it is necessary to find alternatives with fewer side effects and which can be taken over a shorter term with longer lasting results. Proper management of dysmenorrhea will therefore be beneficial to individuals and society.

### SUMMARY

The present disclosure provides software programs, systems, methods that identifies underlying causes of menstrual cycle discomfort using research as described by 246 clinical trials and studies combined with Chinese medicine best practices to provide personalized herbal formula recommendations to standardize care given to individuals seeking an herbal solution to menstrual discomfort. The present disclosure provides an online health assessment based on Traditional Chinese Medicine (TCM) methodologies and practices to recommend herbal formula remedies for women's dysmenorrhea (menstrual pain).

In accordance with some embodiments, the present disclosure provides a computer implemented method. Such a method comprises collecting health data from a female subject (i.e. a woman patient or user), assessing hormonal irregularities based on the health data to identify a pattern of hormonal imbalance, and prescribing a herbal formula based on the pattern of hormonal imbalance to prevent or treat a condition or disorder related to a menstrual cycle of the female subject. The step of collecting health data includes collecting self-reported answers to a list of questions (e.g., about 50-60 questions) by the female subject, and collecting one or more photos of tongue of the female subject. In some embodiments, the health data are collected using an online heath assessment through internet, on a computer, a phone, or any other smart device at the user's (i.e. the female subject's) end. In some other embodiments, the health data may be provided in a practitioner's office during a face-to-face consultation. The condition or disorder includes, but is not limited to, primary dysmenorrhea.

In some embodiments, the pattern of hormonal imbalance includes a cycle pattern such as Qi stagnation, Qi stagnation plus heat, Qi stagnation plus cold, Qi & blood stagnation, Qi & blood stagnation plus heat, and severe Qi & blood stagnation, according to Traditional Chinese Medicine (TCM). The pattern of hormonal imbalance may include a month-long pattern such as Qi stagnation plus heart fire, Qi deficiency plus Qi stagnation, Qi stagnation by stress, blood deficiency, Qi stagnation plus dame-heat, according to Traditional Chinese Medicine (TCM).

The herbal formula is a personalized formulation based on the health data of the female subject in some embodiments. The herbal formulation may include one or more ingredients selected from corydalis, Szechuan lovage, white peony, poria, angelica sinensis, safflower, licorice, skullcap, atractylodes, mint, ginger, gardenia pod, bupleurum, peppermint, moutan cortex, cyperus, motherwort, toosendan fruit, vitex berry, and combinations thereof at a suitable ratio.

The method may further comprise transmitting data for displaying the herbal formula and related information (e.g., key ingredients) to the female subject. The formula and related information may be displayed on a computer, a phone, or any other smart device at the user's end. In some other embodiments, the herbal formula and related information may be displayed at the practitioner's office during a face-to-face consultation.

In another aspect, the present disclosure also provides a computer implemented system. Such a system includes one or more processors, and at least one tangible, non-transitory machine readable medium encoded with one or more programs, to be executed by the one or more processors, to perform the following steps: collecting health data from a female subject, assessing hormonal irregularities based on the health data to identify a pattern of hormonal imbalance, and prescribing a herbal formula based on the pattern of hormonal imbalance to prevent or treat a condition or disorder (e.g., primary dysmenorrhea) related to a menstrual cycle of the female subject.

In some embodiments, the step of collecting health data includes collecting answers to a list of questions by the female subject, and collecting one or more photos of tongue of the female subject. The health data are collected from a user's device using an online heath assessment through internet in some embodiments.

In some embodiments, the function of the one or more programs further comprises transmitting data for displaying the personalized herbal formula and related information (e.g., key ingredients and their function) to the female subject remotely.

### BRIEF DESCRIPTION OF THE DRAWINGS

The present disclosure is best understood from the following detailed description when read in conjunction with the accompanying drawings. It is emphasized that, according to common practice, the various features of the drawings are not necessarily to scale. On the contrary, the dimensions of the various features are arbitrarily expanded or reduced for clarity. Like reference numerals denote like features throughout specification and drawings.

FIG. 1 is a flow chart illustrating an exemplary method in accordance with some embodiments.

FIG. 2 is a block diagram of an exemplary system in accordance with some embodiments.

FIG. 3 shows different exemplary photos of tongues collected as health data from female patients.

FIGS. 4-5 show an example of displaying the health data and the imbalance pattern of a patient.

FIGS. 6-7 show an example of displaying the recommended herbal formula and its ingredients based on the health data and the imbalance pattern of the patient in FIGS. 4-5.

### DETAILED DESCRIPTION

This description of the exemplary embodiments is intended to be read in connection with the accompanying drawings, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” refer to a relationship wherein structures are secured or attached to one another either directly or indirectly through intervening structures, as well as both movable or rigid attachments or relationships, unless expressly described otherwise.

For purposes of the description hereinafter, it is to be understood that the embodiments described below may assume alternative variations and embodiments. It is also to be understood that the specific articles, compositions, and/or processes described herein are exemplary and should not be considered as limiting.

In the present disclosure the singular forms “a,” “an,” and “the” include the plural reference, and reference to a particular numerical value includes at least that particular value, unless the context clearly indicates otherwise. Thus, for example, a reference to “a processor” is a reference to one or more of such structures and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

Because of the problems associated with NSAIDS and hormonal contraceptives currently used, more consumers are seeking natural and holistic remedies for menstrual symptoms. Currently, natural solutions are primarily one-size-fits-all with mixed results of efficacy or prohibitively more expensive option of seeking a practitioner.

It is an emerging trend that many women are seeking alternatives to conventional medicine including herbal medicine. Non-steroidal anti-inflammatory drugs or the contraceptive pill have been used successfully for treatment but more women are looking for non-drug therapies. Chinese herbal medicine has been used in China, and it is currently used in public hospitals in China for the treatment of primary dysmenorrhea. The use of Chinese herbal medicine is promising in reducing menstrual pain in the treatment of primary dysmenorrhea, compared to conventional medicine such as NSAIDs and the oral contraceptive pill, or even acupuncture and heat compression. No significant adverse effects have been identified.

Traditional Chinese Medicine (TCM) has been used to treat diseases and has many beneficial effects for disease control. Herbal formulas are mixtures of several single herbs with certain proportions according to TCM classical literature and have specific indications to TCM syndrome and therapeutic effects. TCM views the human body as a dynamic energy system with yin-yang balance, Qi which flows in good health, and disease as an unbalanced state. While these seem like abstract concepts, the biological effects of herbs have been shown to include affecting the central nervous system, endocrine system, on the activity of sympathetic nerves, basal metabolic rate, the function of organs and tissues, and secretion of cytokines out of the body.

In addition to diagnosis of a western-defined disease (such as dysmenorrhea), TCM doctors use a technique called pattern diagnosis to focus treatments on the subcategories of said disease; for example, blood stasis and cold accumulation of the uterus or blood and qi deficiency syndrome for dysmenorrhea. The TCM syndrome is determined by a TCM doctor on the basis of signs and symptoms displayed by the patient, their physical condition, disease status, and constitution, after which CHM prescriptions for treatment are given. This individualization is a key primary requirement of traditional treatment with CHM. Personalized medicine refers to the right treatment for the right individual at the right time and has the potential to diminish the incidence of drug adverse reactions, eliminate invalid therapy, improve the efficacy of treatments, ultimately, achieve optimal health outcomes.

Analysis of a nation-wide prescription database which covers 98% of the population in Taiwan showed that Chinese herbal medicine (CHM) is well-accepted and commonly used to treat various women's health conditions, such as menopausal symptoms, endometriosis, dysfunctional uterine bleeding, and even dysmenorrhea. Investigations in this database showed that around 90% of women diagnosed with these conditions sought CHM treatment. In total 57,315 prescriptions of herbal medicine for dysmenorrhea were analyzed. In this study, the most common diagnosis by TCM syndrome for primary dysmenorrhea was “Qi stagnation and Blood stasis.” Surprisingly, the presentation and pathogenesis of this TCM syndrome and the current leading theory about primary dysmenorrhea from a western standpoint are remarkably similar.

In TCM teaching, the stabbing and distension pain is caused by impeding blood flow, which may be caused by constitution, emotional stress, and even environmental factors, such as Qi stagnation, Cold, Dampness, and Blood deficiency in TCM's categories. This restricted blood flow in TCM resembles uterine ischemia due to prostaglandin-induced vasoconstriction in western medicine. Furthermore, uterine vascular relaxation can be found when trying to resolve blood stasis via CHM.

The inventors at ZenChi combine modern technology and medical research with traditional Chinese medicine to give more people access to personalized herbal remedies for this problem. Menstrual pain and/or discomfort are triggered by the hormonal imbalances. Zenchi's platform educates people on the underlying imbalance that is triggering their pain, and recommends a personalized herbal formula to address these imbalances that is triggering their symptoms.

The present disclosure provides a computer implemented method and a computer implemented system. In some embodiments, the present disclosure provides a method of assessing hormonal irregularities via an online health assessment, which identifies patterns of imbalances in respect to diagnosis used in Traditional Chinese Medicine (TCM). In some other embodiments, such a method and such a system can be used in the face-to-face consultation in a practitioner's office when a patient goes to meet the practitioner in person.

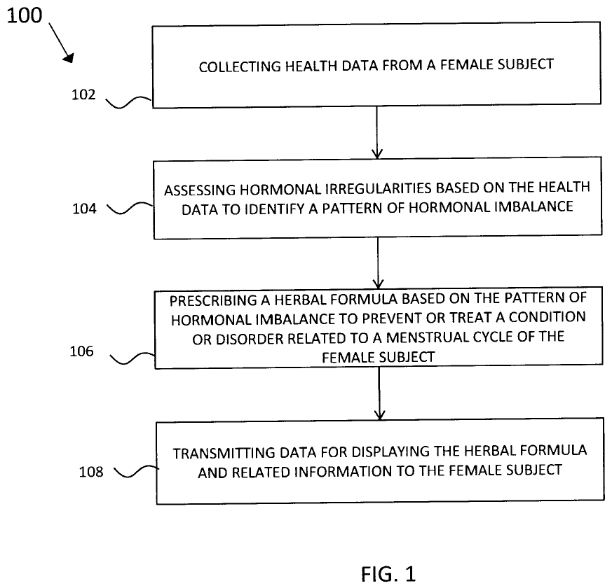

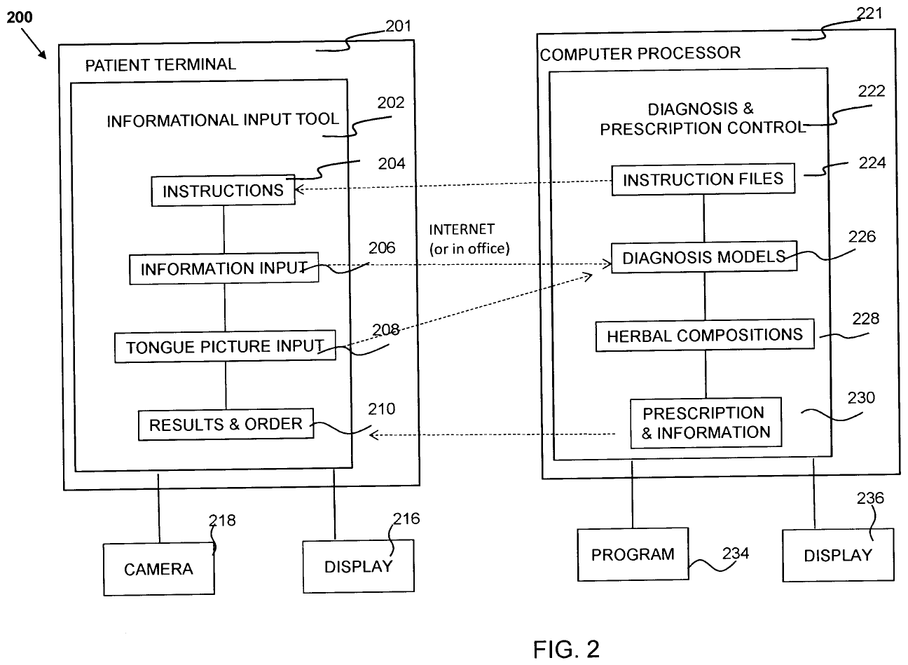

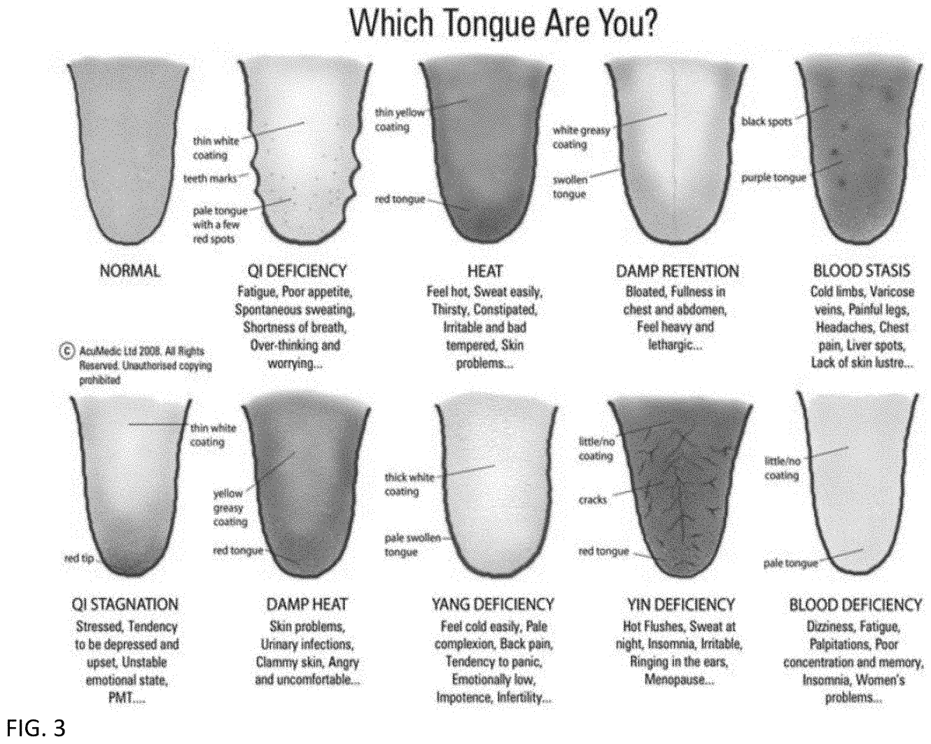

In FIGS. 1-2, like items are indicated by like reference numerals, and for brevity, descriptions of the structure, provided above with reference to the preceding figures, are not repeated. The method described in FIG. 1 is described with reference to the exemplary system, described in FIG. 2 and the examples in FIGS. 3-7.

Referring to FIGS. 1-2, an exemplary method 100 is illustrated. Such a method may be implemented in an exemplary system 200 in accordance with some embodiments.

At step 102 (FIG. 1), health data from a female subject (i.e. a woman patient or user) are collected, for example, through online assessment. This may include collecting self-reported answers to a list of questions (e.g., about 50-60 questions) by the female subject, and collecting one or more photos of tongue of the female subject. In some embodiments, the health data are collected using an online heath assessment through interne, on a computer, a phone, or any other smart device at the user's (i.e. the female subject's) end.

In some embodiments, collecting data promises collecting data based on user's self-reported online health assessment of about 60 questions. The questions may seem unconnected; however, they are linked together and answers on how symptoms are experienced provide valuable input into the diagnostic process and allow the creation of a unique treatment plan for an individual. This taking into account a wide variety of signs and symptoms is the key to the success of TCM and its ability to treat conditions where other methods may have failed.

Questions are guided by TCM lines of questioning, which include asking about all body systems in order to identify the correct pattern. The questions include, but are not limited to, menstrual cycle such as volume of blood, color of blood, pain scale, regular or irregular, vaginal discharge, presence of symptoms such as breast tenderness, fatigue, acne, bloating, headaches, location and quality of pain, and other symptoms. General health inquiries may include topics such as: body temp sensations, sweat, urine, stool, thirst, appetite, sleep, and other factors. The questions about lifestyle may include diet, exercise, stress levels, and other factors.

Practitioners of traditional Chinese medicine have considered the tongue a key clue in any diagnosis. The tongue is the only internal organ visible without a scalpel. The tongue may not seem like an organ in the traditional sense, but it is 100% pure muscle, fed directly by blood and nerves, with cells that regenerate every 10 days (much like the cells lining the digestive tract). Looking at it can give clues about what's happening with the other organs and general internal environment. Specifically, Chinese medicine looks at the color, moisture, shape, and strength of the tongue in order to determine the state of health in regards to the previously mentioned pattern diagnosis. Each pattern has a predefined corresponding color, moisture, shape, and strength of the tongue and can be used to determine or confirm diagnosis.

FIG. 3 illustrates some exemplary photos of tongues, which are used in the method provided in the present disclosure.

Referring back to FIG. 1, at step 104, hormonal irregularities are assessed based on the collected health data to identify a pattern of hormonal imbalance occurred in the female subject. The present disclosure provides a method of assessing hormonal irregularities via an online health assessment, which identifies patterns of imbalances with respect to diagnosis used in Traditional Chinese Medicine (TCM).

As described herein, the pattern of hormonal imbalance includes a cycle pattern such as Qi stagnation, Qi stagnation plus heat, Qi stagnation plus cold, Qi & blood stagnation, Qi & blood stagnation plus heat, and severe Qi & blood stagnation, according to Traditional Chinese Medicine (TCM).

In an average four-week cycle, there are four phases of needs according to TCM. In week 4, the Qi needs to be moved and blood needs to be nourished in order to prepare for menstruation. However, the following cycle imbalance patterns may occur with the related symptoms and characteristic tongues:

(1) Qi Stagnation: This is the base experience of week 4 of the menstrual cycle in TCM. Symptoms are generally mild experience of cycle-related signs and symptoms such as breast tenderness, acne, fatigue, headache, mood swings, and dull pain; Tongue: pink or pale, red sides, thin coating.

(2) Qi Stagnation + Heat: this pattern includes the Qi Stagnation signs + symptoms, in addition to heat symptoms (as it relates to TCM) such as excess thirst, insomnia dark colored urine, tinnitus, running hot, warmth in abdomen during menstruation, night sweats; Tongue: red body or tip, yellow coat.

(3) Qi Stagnation + Cold: this pattern includes the Qi Stagnation signs + symptoms, in addition to cold symptoms (as it relates to TCM) such as cold in abdomen during menstruation, running cold and not being able to warm up with layers, copious pale urine; Tongue: pale, swollen, thick white coat.

(4) Qi & Blood Stagnation: this pattern includes the Qi Stagnation signs + symptoms, but with more prevalent pain symptoms: pain that takes them out of work/school for one day or more, pain that's over 7/10 on the pain scale, sharp stabbing pain, etc.; Tongue: purple or purple spots.

(5) Qi & Blood Stagnation + Heat: this pattern includes the Qi Stagnation + Heat signs & symptoms, in addition to heat symptoms (as it relates to TCM) such as excess thirst, insomnia dark colored urine, tinnitus, running hot, warmth in abdomen during menstruation, night sweats; in addition to more prevalent pain symptoms: pain that takes them out of work/school for one day or more, pain that's over 7/10 on the pain scale, sharp stabbing pain, etc.; Tongue: red body or tip, potential purple spots, yellow coat.

(6) Severe Qi & Blood Stagnation: this pattern includes the Qi & Blood Stagnation + Heat signs & symptoms, in addition to heat symptoms (as it relates to TCM) such as excess thirst, insomnia dark colored urine, tinnitus, running hot, warmth in abdomen during menstruation; in addition to more prevalent pain symptoms: pain that takes them out of work/school for one day or more, pain that's over 7/10 on the pain scale, sharp stabbing pain, etc.; in addition to a stronger experience of the qi stagnation symptoms, such as more severe, frequent, or impactful experience of breast tenderness, mood swings, acne, fatigue, etc.; Tongue:

red body or tip, potential purple spots, yellow coat.

The pattern of hormonal imbalance may also include a following month-long blend pattern such as Qi stagnation plus heart fire, Qi deficiency plus Qi stagnation, Qi stagnation by stress, blood deficiency, Qi stagnation plus dame-heat, according to Traditional Chinese Medicine (TCM):

(7) Qi deficiency + heart fire: Memory loss, forgetfulness, absent-mindedness, lack of concentration, menorrhagia, palpitations, insomnia (difficulty falling asleep), nightmares, reduced appetite, rale complexion, pale lips and nails, early menstruation with copious pale blood, irregular menstruation, premenstrual spotting, excess perspiration, night sweats, Leukorrhea, dizziness, blurred vision, spots before the eyes, bruising easily, low spirits, depression, anxiety/phobias/panic attacks/fear, fatigue and lethargy, weakness, abdominal distention after eating, dry skin and hair; Tongue: pale, maybe swollen, white coat.

(8) SP Qi deficiency + LV Qi stagnation (digestive aid): Distention and fullness in the chest and epigastrium, abdominal distention with occasional pain, belching, acid regurgitation, nausea and vomiting, aversion to food/lack of appetite, maybe diarrhea, maybe constipation, bad breath, Stress; Tongue: pink with potential yellow thick coat.

(9) Qi stagnation (stress): Hypochondriac pain and distention, alternating fever and chills, depression, easily angered, irritability, chest fullness, bitter taste in mouth, fidgeting, abdominal pain and cramps, breast distention and pain, dysmenorrhea and/or irregular menstruation, or amenorrhea; Tongue: pink, thin and white coat.

(10) Blood deficiency (cycle recovery): Dizziness, blurred vision, pale complexion, pale nails, pale lips, generalized muscle tension, tremors and tics, potential irregular menstruation with little flow or menorrhagia with pale blood, amenorrhea or delayed menstruation, lower abdominal pain, vertigo, tinnitus, blurred vision, insomnia, spotting of pale blood, dry hair, palpitations, dizziness, dry skin, potential leukorrhea; Tongue: pale.

(11) Qi stagnation + damp-heat (acne): Temper/mood swings, red eyes, bitter taste in the mouth, painful urination or UTI, yellow leukorrhea, red painful swollen acne related to menstrual cycle, pain in the armpits; Tongue: red or red edges, yellow or greasy coat.

These define the pattern based in TCM philosophy by a unique and proprietary scoring method based on 1 year in beta and analysis of 1200+unique health assessments from beta users.

At step 106, a herbal formula is prescribed or recommended based on the pattern of hormonal imbalance to prevent or treat a condition or disorder (e.g., dysmenorrhea) related to a menstrual cycle of the female subject.

The herbal formula is a personalized formulation based on the health data of the female subject in some embodiments. The herbal formulation may include one or more ingredients selected from corydalis, Szechuan lovage, white peony, poria, angelica sinensis, safflower, licorice, skullcap, atractylodes, mint, ginger, gardenia pod, bupleurum, peppermint, moutan cortex, cyperus, motherwort, toosendan fruit, vitex berry, and combinations thereof at a suitable ratio.

Corydalis (Yan Hu Suo) is analgesic, anti-inflammatory, and sedative. Corydalis is a plant species in the genus Corydalis. Its tube is used.

Szechuan lovage (Chuan Xiong) reduces spasms, pain, & inflammation.

White Peony Root (Bai Shao) is a blood builder, pain reliever, and a spasm reducer.

Poria (Fu Ling) is energizing and balancing.

Angelica Sinensis (Dang Gui) regulates hormones, and is anti-inflammatory.

Safflower (Hong Hua) increases blood flow and decreases inflammation.

Skullcap (Huang Qin) reduces insomnia, pain, and anxiety. Skullcap (Scutellaria lateriflora) is a flowering perennial plant.

Atractylodes (Bai Zhu) increases mental clarity, and is balancing & energizing. Atractylodes is a genus of Asian flowering plants in the sunflower family. A species is Atractylodes macrocephala.

Ginger (Pao Jiang) is anti-inflammatory and a GI regulator.

Bupleurum (Chai Hu) is anti-inflammatory, and is an antioxidant and a pain reliever. Bupleurum is also a plant. Its root may be used.

Peppermint (Bo He) is a mood regulator, an anti-inflammatory agent, and a pain reliever.

Licorice (Gan Cao) is anti-inflammatory, and is a GI regulator and a formula balancer. Liquorice or licorice is the root of Glycyrrhiza glabra, from which a sweet flavor can be extracted. The liquorice plant is an herbaceous perennial legume native to the Middle East, southern Europe, and parts of Asia, such as India.

Moutan Cortex (Mu Dan Pi) is a pain reliever, is anti-inflammatory and balancing.

Gardenia Pod (Zhi Zi) is anti-inflammatory, and a mood regulator.

Cyperus (Xiang Fu) is a hormone regulator, an antioxidant, and a pain reliever. Cyperus totundus is known in Chinese as xiangfu or xiangfuzi.

Motherwort (Yi Mu Cao) is an uterine soother, is anti-inflammatory and antioxidant.

Toosendan Fruit (Chuan Lian Zi) is anti-inflammatory, and is pain reliever and a spasm reducer.

Vitex berry is a hormone and stress modulator, an antioxidant, and an anti-inflammatory agent.

The herbs can be sourced from the regions that are traditionally known for the most potency, i.e. from where they have been grown on those mountainsides for thousands of years. Most of these regions are in China and greater Asia.

Referring to FIG. 1, the exemplary method 100 may include step 108 for transmitting data for displaying the herbal formula and related information (e.g., key ingredients) to the female subject. The formula and related information may be displayed on a computer, a phone, or any other smart device at the user's end. In some other embodiments, the herbal formula and related information may be displayed at the practitioner's office during a face-to-face consultation if a female subject visits there.

Referring to FIG. 2, an exemplary system 200 is illustrated. Such a system 200 includes one or more processors 221, and at least one tangible, non-transitory machine readable medium encoded with one or more programs 234, to be executed by the one or more processors, to perform the functions or the method as described above. The processor(s) 221 may include a diagnosis and prescription control 222, which includes instruction files 224, diagnosis models 226, herbal formula/composition database 228, and a prescription and information module 230. The processors 221 may be connected with one or more displays 238.

Through internet, the one or more processors 221 may be communicated with a patient terminal 201, which may be a computer, a cell phone, or any other smart device at a user's end. Through the one or more programs 234, health data from a female subject may be collected through an information input tool 202 such as a web page or program. The patient terminal 201 may be equipped with a display 216 for showing the instructions and end results, and a camera 218 for taking photos.

A user such as a female patient may review instructions 204 received from the processors 221, and provide health information input 206 by answering the list of questions received from the processors 221 (step 102 of FIG. 1). The user and the female patient may be the same person or different people. The user may further provide one or more photos of the tongue of the female subject as the tongue picture input 208. The photos may be taken from the camera 218. The health data from the female subject are sent to the processors 221 at step 102 of FIG. 1.

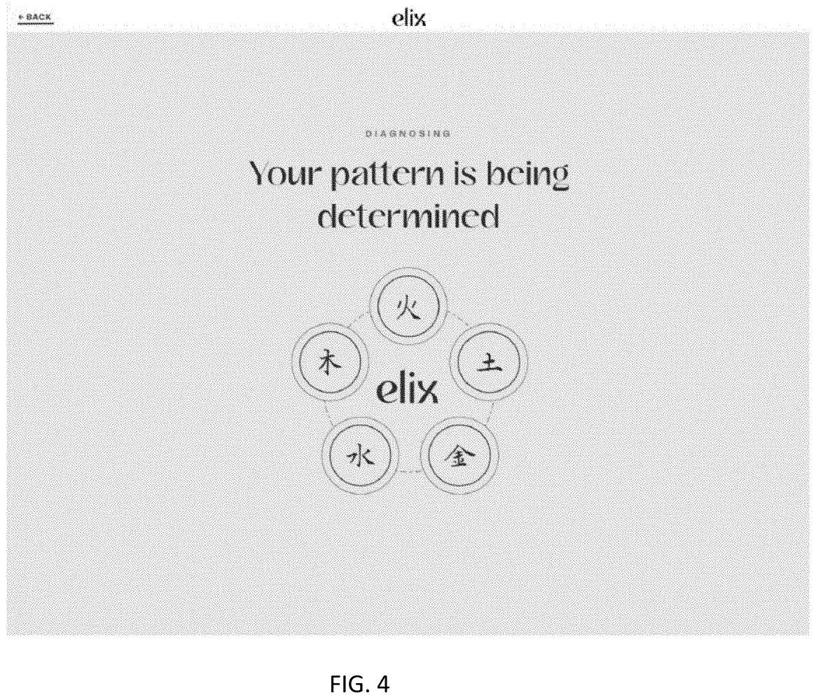

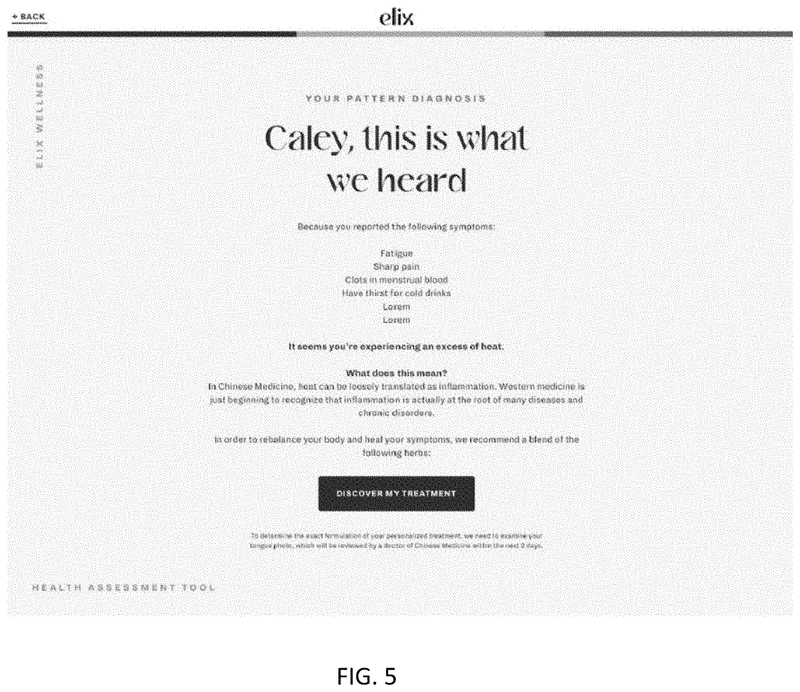

Referring to FIG. 2, through the diagnosis models 226, hormonal irregularities are assessed based on the health data to identify a pattern of hormonal imbalance at step 104 of FIG. 1. Referring to FIG. 4, a message may be sent to a user (e.g., a patient named Caley) showing that the patter is being determined during this step. Referring to FIG. 5, the symptom summary and the pattern diagnosis for the patient (e.g., Caley) can be displayed on the user's end.

At step 106 of FIG. 1, an herbal formula can be then recommended or prescribed based on the pattern of hormonal imbalance to prevent or treat a condition or disorder (e.g., primary dysmenorrhea) related to a menstrual cycle of the female subject. Herbal formula database 228 may include information about different herbal formulas corresponding to different imbalance patterns. The recommendation or prescription, including detailed information such as formula and ingredients, is provided in the prescription and information module 230.

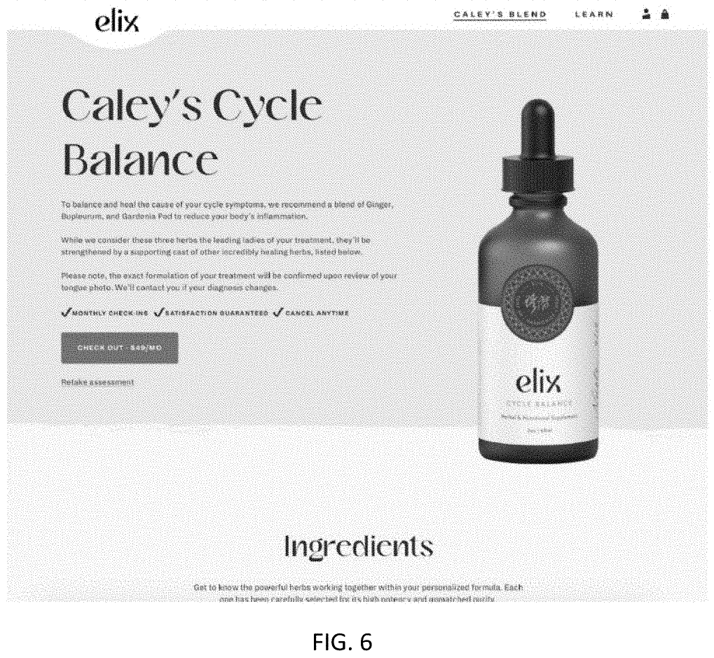

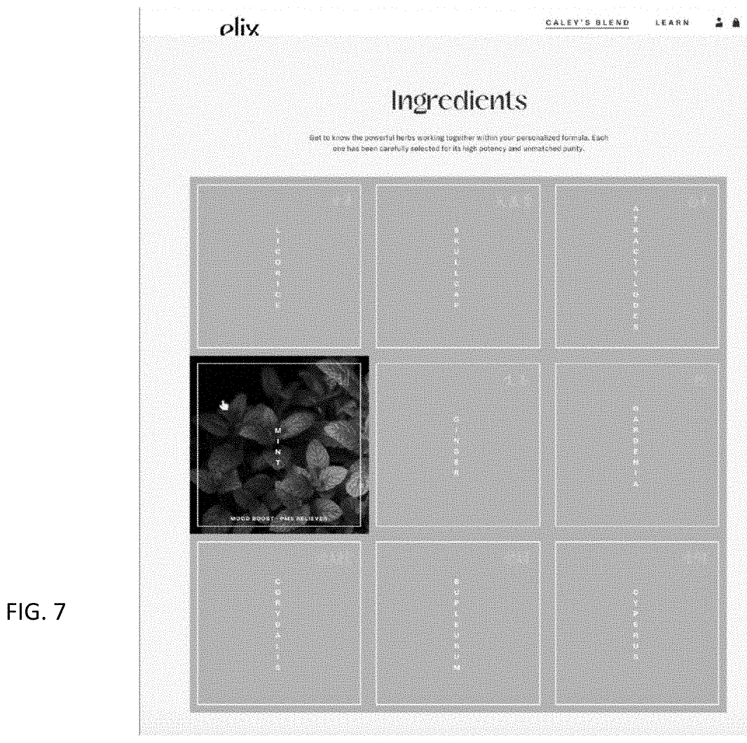

At step 108, the data for such a recommendation or prescription may be transmitted to the user terminal 201. The personalized herbal formula and related information (e.g., key ingredients and their function) may be displayed to the female subject remotely. Referring to FIGS. 6-7, the recommendation and prescription including herbal ingredients are displayed on the user's end. These include the herbal recommendations and ‘personalized’ output experience for Caley as illustrated in FIGS. 6-7. For example, the personalized herbal formula for Caley includes licorice, skullcap, atractylodes, mint, ginger, gardenia, corydalis, bupleurum, and cyperus at a certain ratio. When Caley pressed the icon for each ingredient, further information about the specific ingredient can be shown.

In the existing Chinese medicine for dysmenorrhea, the approach is “one size fits all,” which is not the most effective, even though it is natural.

For the Chinese herbal medicine for dysmenorrhea, personalized Chinese herbal formulae resulted in significant improvements in pain relief (18 RCTs; RR 2.06, 95% CI 1.80 to 2.36), overall symptoms (14 RCTs; RR 1.99, 95% CI 1.65 to 2.40) and use of additional medication (5 RCTs; RR 1.58, 95% CI 1.34 to 1.87) after up to three months of follow-up when compared to commonly used OTC (one size fits all) Chinese herbal health products. “RCT” represents randomized control trials. “CI” represents confidence interval. “RR” represents relative risk.

In the present disclosure, the method, the software and the system provided focus on treating menstrual health and cycle regulation, and on herbal supplement regimen other than behavioral therapy. The Chinese medicine pattern diagnosis is used as the basis of the assessment software. Cycle-related symptoms are the reproductive system's way of signaling to the outside that something is wrong. This can be displayed as painful periods, be linked to hormonal imbalances, and/or affect fertility because of how they impact uterine function. Infertility can correspond to gynecological conditions such as endometriosis, uterine fibroid, adenomyosis, etc. However, women with mild conditions may not seek treatment but even if they do, these conditions can be hard to diagnose. This means that many times symptoms that are considered normal or idiopathic, can negatively impact fertility. With respect to fertility treatment, not the basal body temperature or fertility charting is used. Therefore, a ‘daily recording’ by the user is not required. The method in the present disclosure, which is only a monthly assessment, targeting a reduction in undesired symptoms rather than an “ideal fertility state.” In the present disclosure, the inventors track a unique set of patterns and associated symptoms from both traditional teachings and modern research, using a unique set of herbs to treat said patterns. Because of these features above, the algorithm for treatment assignment is different.

### EXPERIMENTAL TESTING

Experimental testing has been conducted with beta users. The beta users (%) initially reported: 98% with pain, 86% with bloating, 85% with fatigue, 84% with mood swings, 72% with breast tenderness, 71% with headaches, 65% with acne, 53% with loose stools, and 44% with constipation. Of the beta participants, 18% had endo; 10% had PCOS, 8% had fibroids.

Of the beta participants, 50% used the suggested treatments for up to 4 months, 50% used the treatments for 5 months to 1 year and over. 26% beta users were on hormonal birth control (55% on for 5+ years//62% to control cycle-related symptoms). 6% of the beta users were regularly using painkillers during or before beta.

As a result, 93% of users reported a positive improvement in pain and/or symptoms after the first month, while the majority felt the biggest improvements after 3 months.

Following one-month treatment, symptomatic improvements were shown as below:

Pain, 88% felt some improvement of pain;

Complete relief of

bloating, 32%;

fatigue, 54%;

mood swings, 46%;

breast tenderness, 29%;

headaches, 42%;

acne, 21%;

loose stools, 23%; and

constipation, 30%.

On average, highest efficacy achieved after 3 months.

The system and the method provided in the present disclosure have significant advantages. For example, data from electronic communications provides the best combination of supplements. The personalized prescription and recommendation have proved with high efficacy.

The methods and system described herein may be at least partially embodied in the form of computer-implemented processes and apparatus for practicing those processes. The disclosed methods may also be at least partially embodied in the form of tangible, non-transient machine readable storage media encoded with computer program code. The media may include, for example, RAMs, ROMs, CD-ROMs, DVD-ROMs, BD-ROMs, hard disk drives, flash memories, or any other non-transient machine-readable storage medium, or any combination of these mediums, wherein, when the computer program code is loaded into and executed by a computer, the computer becomes an apparatus for practicing the method. The methods may also be at least partially embodied in the form of a computer into which computer program code is loaded and/or executed, such that, the computer becomes an apparatus for practicing the methods. When implemented on a general-purpose processor, the computer program code segments configure the processor to create specific logic circuits. The methods may alternatively be at least partially embodied in a digital signal processor formed of application specific integrated circuits for performing the methods.

Although the subject matter has been described in terms of exemplary embodiments, it is not limited thereto. Rather, the appended claims should be construed broadly, to include other variants and embodiments, which may be made by those skilled in the art.

What is claimed is:

1. A computer implemented method comprising:
collecting health data from a female subject;
assessing hormonal irregularities based on the health data to identify a pattern of hormonal imbalance; and
prescribing a herbal formula based on the pattern of hormonal imbalance to prevent or treat a condition or disorder related to a menstrual cycle of the female subject.

2. The method of claim 1, wherein collecting health data includes collecting answers to a list of questions by the female subject, and collecting one or more photos of tongue of the female subject.

3. The method of claim 2, wherein the health data are collected using an online heath assessment through internet.

4. The method of claim 2, wherein the list of questions includes about 60 questions.

5. The method of claim 1, wherein the condition or disorder is primary dysmenorrhea.

6. The method of claim 1, wherein the pattern of hormonal imbalance includes a cycle pattern selected from the group consisting of Qi stagnation, Qi stagnation plus heat, Qi stagnation plus cold, Qi & blood stagnation, Qi & blood stagnation plus heat, and severe Qi & blood stagnation, according to Traditional Chinese Medicine (TCM).

7. The method of claim 1, wherein the pattern of hormonal imbalance includes a month-long pattern selected from the group consisting of Qi stagnation plus heart fire, Qi deficiency plus Qi stagnation, Qi stagnation by stress, blood deficiency, Qi stagnation plus dame-heat, according to Traditional Chinese Medicine (TCM).

8. The method of claim 1, wherein the herbal formula is a personalized formulation based on the health data of the female subject.

9. The method of claim 8, wherein the herbal formulation includes one or more ingredients selected from the group consisting of corydalis, Szechuan lovage, white peony, poria, angelica sinensis, safflower, licorice, skullcap, atractylodes, mint, ginger, gardenia pod, bupleurum, peppermint, moutan cortex, cyperus, motherwort, toosendan fruit, vitex berry, and combinations thereof.

10. The method of claim 1, further comprising transmitting data for displaying the herbal formula and related information to the female subject.

11. A computer implemented system comprising:
one or more processors; and
at least one tangible, non-transitory machine readable medium encoded with one or more programs, to be executed by the one or more processors, to perform steps of:
collecting health data from a female subject;
assessing hormonal irregularities based on the health data to identify a pattern of hormonal imbalance; and
prescribing a herbal formula based on the pattern of hormonal imbalance to prevent or treat a condition or disorder related to a menstrual cycle of the female subject.

12. The system of claim 11, wherein collecting health data includes collecting answers to a list of questions by the female subject, and collecting one or more photos of tongue of the female subject.

13. The system of claim 12, wherein the health data are collected from a user's device using an online heath assessment through internet.

14. The system of claim 11, wherein the condition or disorder is primary dysmenorrhea.

15. The system of claim 11, wherein function of the one or more programs further comprises transmitting data for displaying the herbal formula and related information to the female subject.
